(19) United States
(12) Patent Application Publication
(10) Pub. No.: US 2023/0263528 A1
(43) Pub. Date: Aug. 24, 2023

(54) INTRASACULAR FLOW DIVERTER AND RELATED METHODS

(71) Applicant: NV MEDTECH, Inc., San Clemente, CA (US)

(72) Inventor: Michael Jones, Gardnerville, NV (US)

(21) Appl. No.: 18/113,598

(22) Filed: Feb. 23, 2023

Related U.S. Application Data
(60) Provisional application No. 63/313,652, filed on Feb. 24, 2022.

(51) Int. Cl. A61B 17/12

(52) U.S. Cl. CPC A61B 17/12113; A61B 17/12022

(57) ABSTRACT

An intrasacular flow diverter is provided. The intrasacular flow diverter includes a plurality of wires coiled to form a collapsible, substantially spherical frame configured to expand within, and substantially conform to a shape of an inside surface of, the aneurysm. The intrasacular flow diverter includes a membrane disposed on at least a portion of the outer surface of the substantially spherical frame. The membrane includes a first portion configured to be disposed directly against a neck of the aneurysm. The membrane includes a second portion configured to be disposed within the aneurysm and distal to the neck of the aneurysm. The first portion has a first porosity to blood flow and the second portion has a second porosity to blood flow greater than the first porosity. Related methods of use and manufacturing are also provided.

### RELATED APPLICATIONS

This application claims priority to U.S. Provisional Application 63/313,652, the entire disclosure of which is hereby incorporated by reference in its entirety.

### BACKGROUND

Intracranial aneurysms are among the most serious of medical conditions. Their typical size and location make them especially difficult to detect and treat; but even small ones, if ruptured, can cause debilitating physical and cognitive impairment, coma, and death. Initial treatment methods involved clip ligation of the neck of the aneurysm in open surgical procedures. More recently, minimally invasive endovascular techniques have been developed. Given the clinical significance of the condition and the difficulties encountered in addressing it, treatment for intracranial aneurysms remains an especially active area of device and surgical procedure development.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

In one embodiment, an intrasacular flow diverter for treating an aneurysm of an intracranial blood vessel comprises one or more wires forming a frame, wherein the frame has a collapsed configuration and an expanded configuration, wherein the frame is configured to transition in use from the collapsed configuration to a deployed configuration that substantially conforms to a shape of an inside surface of a sac of the aneurysm. The intrasaccular flow diverter further comprises an electrospun cover disposed on at least a portion of the frame. The cover comprises a first portion configured to be disposed against a neck of the aneurysm, the first portion of the cover having pores formed therein defining a first porosity thereof, and a second portion having a second porosity to blood flow greater than the first porosity, the second portion configured to be disposed within the sac of aneurysm distal to the neck of the aneurysm.

In another implementation, a method of utilizing an intrasacular flow diverter to treat an aneurysm of a blood vessel comprises disposing the intrasacular flow diverter within a microcatheter in a collapsed state, threading the microcatheter through the blood vessel to a location within the aneurysm, and removing the intrasacular flow diverter from a distal end of the microcatheter such that a plurality of coiled wires forming a collapsed substantially spherical frame of the intrasacular flow diverter expand sufficiently within the aneurysm. In this embodiment, a first portion of a membrane disposed on at least a portion of the outer surface of the substantially spherical frame is disposed directly against a neck of the aneurysm, the first portion having a first porosity to blood flow, and a second portion of the membrane is disposed within the aneurysm and distal to the neck of the aneurysm, the second portion having a second porosity to blood flow greater than the first porosity.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1A illustrates a portion of a blood vessel having an aneurysm, in accordance with some embodiments;

FIG. 1B illustrates an intrasaccular flow diverter located inside of an aneurysm.

FIG. 1C illustrates an embodiment of an intrasaccular flow diverter.

FIG. 2 illustrates a microcatheter disposed within the blood vessel and into the aneurysm, in accordance with some embodiments;

FIG. 3 illustrates an intrasacular flow diverter disposed within the aneurysm within the blood vessel, in accordance with some embodiments;

FIG. 4A illustrates an embodiment of the intrasacular flow diverter of FIG. 3, in accordance with some example embodiments;

FIG. 4B illustrates the intrasacular flow diverter of FIG. 4A but viewed from a different angle from that of FIG. 4A, in accordance with some example embodiments;

FIG. 4C illustrates yet another embodiment of the intrasacular flow diverter of FIG. 3, in accordance with some example embodiments;

FIG. 4D illustrates yet another embodiment of the intrasacular flow diverter of FIG. 3, in accordance with some example embodiments;

FIG. 5A illustrates a cross-section of a collapsed embodiment of the intrasaccular flow diverter of FIG. 3, in accordance with some example embodiments;

FIG. 5B illustrates a cross-section of another collapsed embodiment of the intrasaccular flow diverter of FIG. 3, in accordance with some example embodiments;

FIG. 5C illustrates a cross-section of yet another collapsed embodiment of the intrasaccular flow diverter of FIG. 3, in accordance with some example embodiments;

FIG. 5D illustrates a cross-section of yet another collapsed embodiment of the intrasaccular flow diverter of FIG. 3, in accordance with some example embodiments;

FIG. 6 illustrates a flowchart related to a method of using an intrasacular flow diverter, in accordance with some example embodiments; and

FIG. 7 illustrates a flowchart related to a method of manufacturing an intrasacular flow diverter, in accordance with some example embodiments.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

### Definitions

Collapsed Configuration— A device is in a collapsed configuration when it is sheathed proximate to and inside a distal end of a catheter ready for use in an endovascular surgical procedure.

Expanded Configuration— A device is in an expanded configuration when unsheathed outside the vasculature such that outward expansion of the sidewall is unconstrained by any surrounding walls. For flow diverters that are manually expanded by the surgeon, the expanded configuration is obtained when the flow diverter is manually expanded to its maximum intended diameter for normal use.

Deployed Configuration— A device is in the deployed configuration when unsheathed and with its side wall in contact with the inner wall of a vessel. A deployed configuration may have a smaller diameter for the sidewall than an expanded configuration depending on the size of the vessel in which the device is deployed. A device in the deployed configuration is typically close to but not fully in the expanded configuration.

Frame—One or more struts forming a structural scaffold defining a device sidewall configured to conform to the inner surface of a vessel segment when the device is in a deployed configuration. An arrangement of metal wires is a common implementation of struts for a frame.

Frame Porosity—The fractional open area of a selected portion of the sidewall defined by the struts of the device when the device is in a deployed configuration. The frame porosity may vary in different portions of a sidewall. Thus, for a given selected portion of a frame sidewall, the frame porosity is the total area of a selected portion of a sidewall minus the area of the struts defining the selected portion of the sidewall, divided by the total area of the selected portion of the sidewall when the device is in an expanded configuration.

Cover— A film or membrane connecting two or more struts of a frame and extending over some or all of the open area of the sidewall defined by the struts of the frame.

Cover Porosity—The fractional open pore area of a selected portion of a cover membrane when the device is in an expanded configuration. Cover porosity may vary in different portions of a sidewall that is covered by the membrane.

Pore Size— The size of a given pore is defined to be the diameter of an inscribed circle with three points of contact with the actual boundary of the given pore.

Cover Porosity Distribution—The cover porosity in a selected region of a cover may be distributed among groups of pores having particular pore characteristics, usually size characteristics. For example, a cover with 30% porosity may have a certain fraction of that porosity contributed by pores with a defined size range. The cover porosity distribution refers to a characterization of the amount of total porosity contributed by pores with a defined set of one or more properties.

Cover Permeability— Cover permeability is a qualitative or quantitative measure of the ability of different substances to pass through the cover when the device is in normal use in a vessel. Cover permeability is affected by several different aspects of a cover, including cover porosity and porosity distribution with respect to different size components of blood and/or particles in blood as well as the chemical properties of the cover material with respect to the chemical properties of different components of blood and/or particles in blood. Cover permeability to various blood components can also be affected by local flow and pressure conditions at the site of implantation in a vessel. The characteristics of the fluid flows encountered during use of the devices described herein where porosity and permeability are relevant device properties will be apparent from the context, and typically involve blood flow through the otherwise unobstructed ostia of intracranial blood vessels and/or necks of intracranial aneurisms over or across which the device is to be applied.

Electrospinning— A technique for depositing a layer of fibers onto a target surface that involves expelling a jet of polymer solution from an orifice in a reservoir to the target surface under the influence of an electric field. By moving the orifice and/or the target surface during the electrospinning process, polymer fibers and fibrous polymer layers and mats having a variety of characteristics can be created. A fiber or fibrous polymer layer or mat so deposited is referred to herein as “electrospun.” A variety of electrospinning techniques and materials suitable for electrospinning are described in paragraphs [0061] to

of U.S. Patent Publication 2018/0161185 to Kresslein et al., which paragraphs are incorporated herein by reference.

### DESCRIPTION OF CERTAIN EMBODIMENTS

The present disclosure relates to intrasacular flow diverters and related methods of using and/or manufacturing the same. In some embodiments, intrasacular flow diverters described anywhere herein may also be considered intrasacular neuro flow diverters, configured at least for application in the tortious blood vessels of the brain. Several example embodiments that overcome many limitations of current devices will now be described in connection with one or more figures.

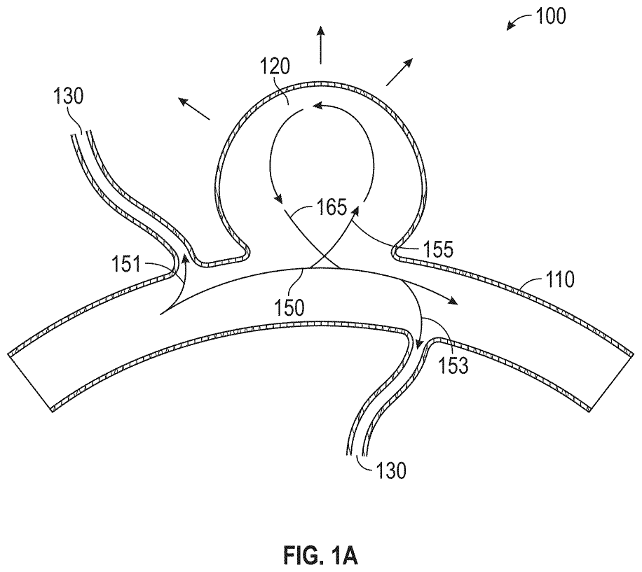

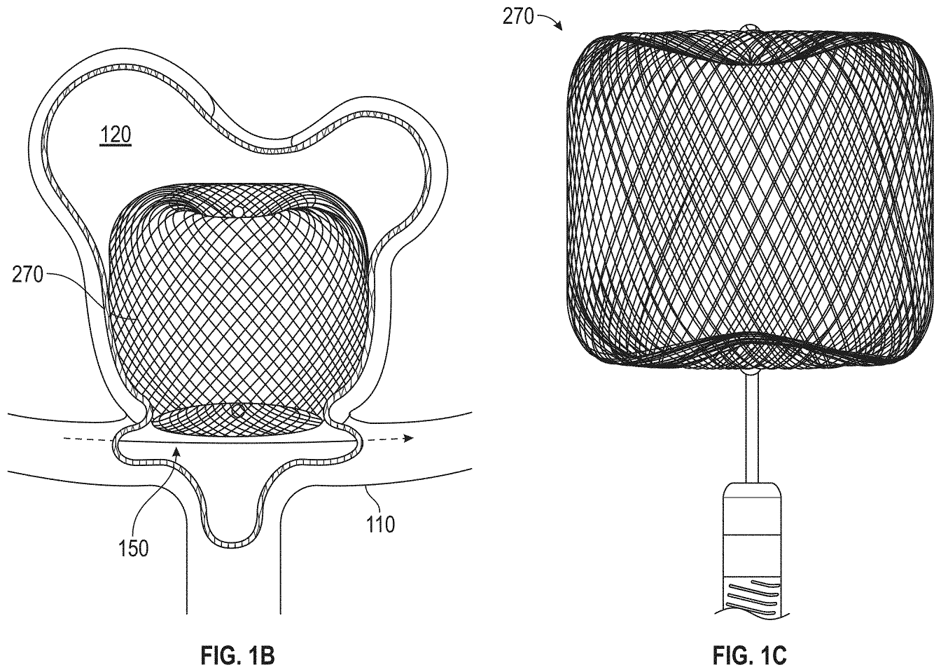

FIG. 1A illustrates a portion of a blood vessel 100. Artery 100 is illustrated as including one or more branching blood vessels 130 and an aneurysm 120, shown bulging beyond adjacent portions of blood vessel 100. FIG. 1A illustrates blood flow through the vessel 110 in the region around the aneurysm 120. In many cases, especially for wider necked aneurysms, some of the vessel blood flow 150 past the aneurysm neck is diverted into the aneurysm as aneurysm inflow 155, where it may circulate backwards and then re-enter the vessel flow 150 as aneurysm outflow 165. This intrasaccular circulation pushes outward on the aneurysm wall, causing expansion and possibly rupture. Endovascular repair of fusiform aneurysms or bifurcation aneurysms is difficult due to complex flow geometries, side branching blood vessels at the bifurcation, and the potential for perforating blood vessels with blood flows 151, 53 that are desirable to preserve.

Endovascular repair of sidewall or bifurcation aneurysms are not always amenable to standard treatments. One such class of aneurysms can include those having particularly wide neck anatomies. Typical treatments for some such types of aneurysms involve the use of a balloon to support and block flow in the parent blood vessel and associated aneurysm while coiling of the aneurysm is accomplished. Traditionally, coils have been used to fill the aneurysm sac due to their inherent softness. However, such coils lack sufficient retention force in large neck aneurysms. Another difficulty can be posed by nearby branching vessels 130, which may prevent the use of a conventional fluid impermeable stent in the vessel 110 across the neck of the aneurysm 120.

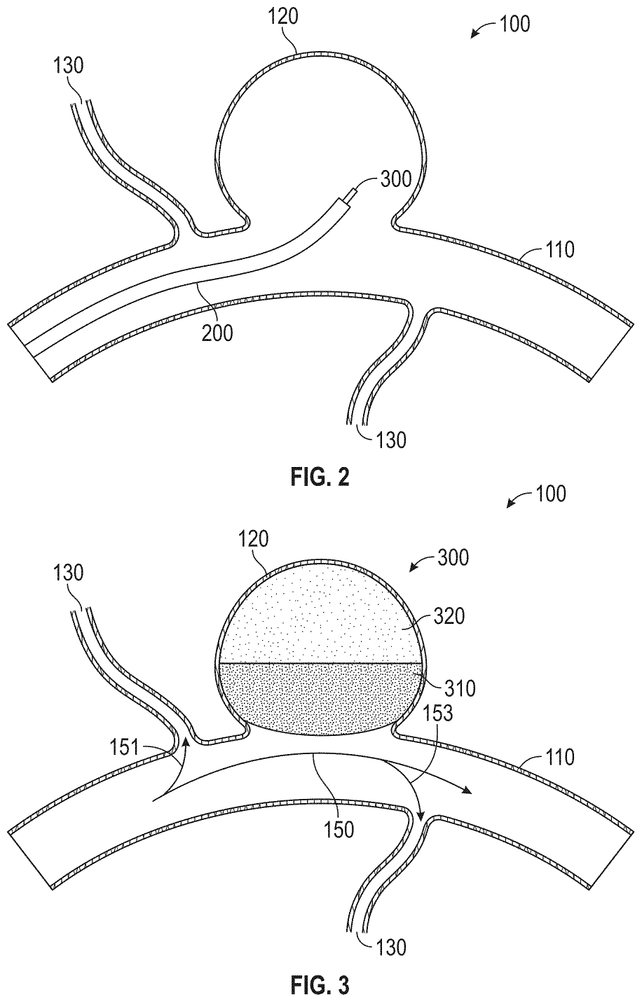

One option is to deploy an intrasaccular occluder 270 into the sac of the aneurysm. This is illustrated in FIG. 2A. Microvention's WEB device, a dense braid of super-elastic nitinol wires that deploys into a 3D shape configured for insertion into the aneurysm sac is one example of such a device, shown in FIG. 2B. However, the WEB device is constructed from dozens of wires to form the braid, which makes it a very stiff device to manipulate during its initial deployment, before enough of the device is out of the microcatheter and able to begin blossoming and, thereby, begin softening. These devices attempt to exclude the aneurysm from the general circulation, creating a clot as a consequence of stasis within the aneurysm.

In some embodiments, an intrasacular flow diverter for treating an aneurysm of a blood vessel is provided. The intrasacular flow diverter includes a plurality of wires coiled to form a collapsible frame configured to expand within, and substantially conform to a shape of an inside surface of, the aneurysm. The intrasacular flow diverter includes a membrane disposed on all or some of the outer surface of the frame. The membrane includes a first portion configured to be disposed directly against a neck of the aneurysm. The membrane may include a second portion configured to be disposed within the aneurysm and distal to the neck of the aneurysm. The first portion has a first porosity to blood flow and the second portion has a second porosity to blood flow greater than the first porosity. Common locations for intracranial aneurysms include the communicating arteries, the internal carotid arteries, and the middle cerebral artery. The devices described herein can, for example, be used in these arteries.

In some other embodiments, a method for utilizing an intrasacular flow diverter to treat an aneurysm of a blood vessel is provided. The method includes disposing the intrasacular flow diverter within a microcatheter in a collapsed state. The method includes threading the microcatheter through the blood vessel to a location within the aneurysm. The method includes removing the intrasacular flow diverter from a distal end of the microcatheter such that a plurality of coiled wires forming a collapsed substantially spherical frame of the intrasacular flow diverter expand sufficiently within the aneurysm that a first portion of a membrane disposed on an entire outer surface of the substantially spherical frame is disposed directly against a neck of the aneurysm, and a second portion of the membrane is disposed within the aneurysm and distal to the neck of the aneurysm. The first portion has a first porosity to blood flow and the second portion has a second porosity to blood flow greater than the first porosity.

In some other embodiments, a method of manufacturing an intrasacular flow diverter configured for treating an aneurysm of a blood vessel is provided. The method includes coiling a plurality of wires to form a collapsible, substantially spherical frame configured to expand within, and substantially conform to a shape of an inside surface of, the aneurysm. The method includes disposing a membrane on all or part of an outer surface of the frame such that the membrane comprises a first portion configured to be disposed directly against a neck of the aneurysm, and a second portion configured to be disposed within the aneurysm and distal to the neck of the aneurysm. The first portion has a first porosity to blood flow and the second portion has a second porosity to blood flow greater than the first porosity.

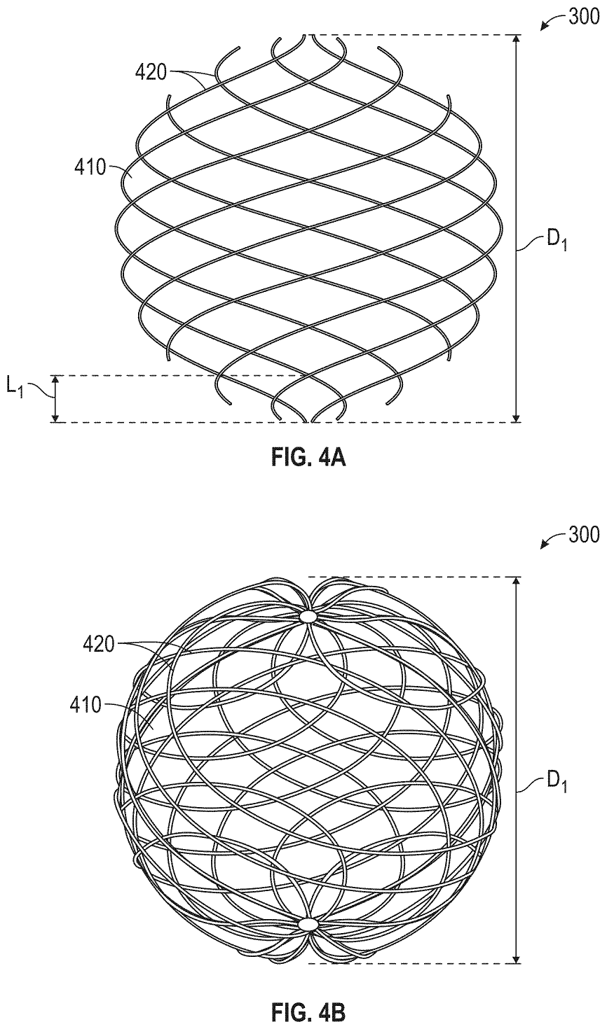

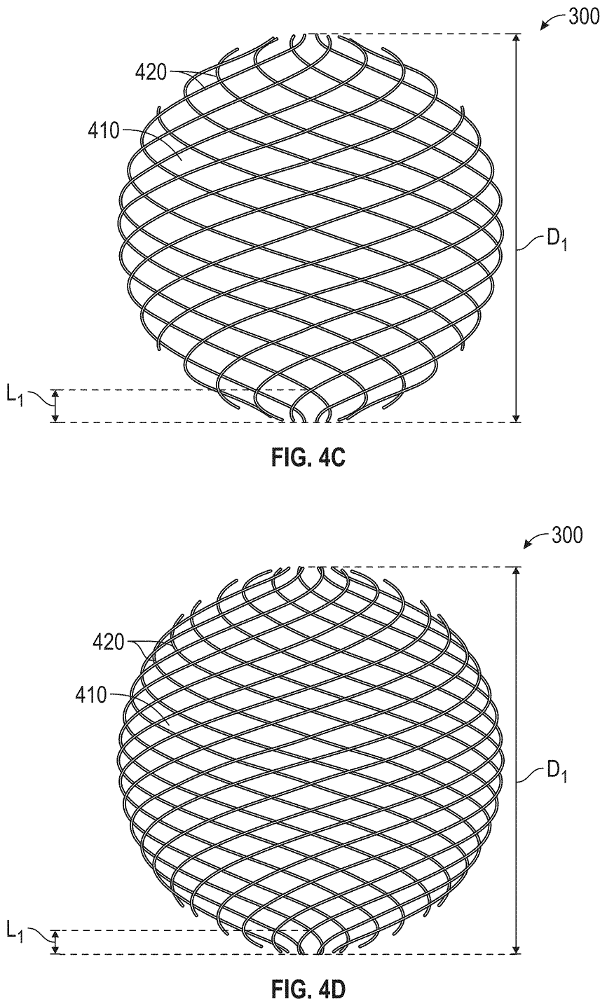

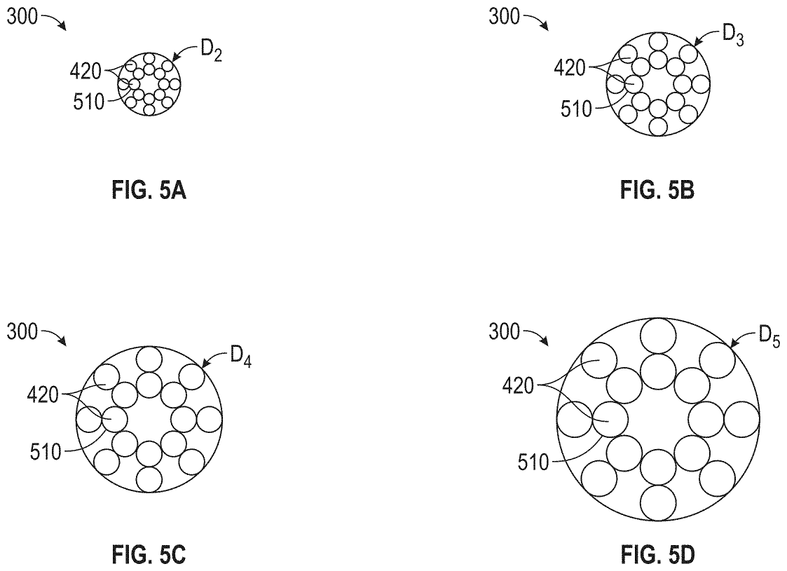

Improvements to intrasaccular flow diverters as described herein may include a self-expanding frame of wires 420 that is configured to fill an aneurysm sac and aid in reconstruction of the neck of aneurysm 120 to, thereby, prevent further growth of aneurysm 120 (see, e.g., any of FIGS. 4A-4D). In some embodiments, each of wires 420 is covered in a polymer 510 configured to minimize metal exposure to the blood (see, e.g., any of FIGS. 5A-5D) and the entire frame of wires 420 is further covered with a polymeric membrane 410 having one or more portions 310, 320 having one or more respective desired porosities (see, e.g., FIG. 3). Particulars of such intrasacular flow diverters 300 will be described in more detail in connection with at least FIGS. 3-5C.

Regarding deployment as illustrated in FIG. 2, such a flow diverter 300 may be disposed within a microcatheter 200 in a collapsed state and the microcatheter 200 threaded through blood vessel 100 until a distal end of microcatheter 200 is disposed within aneurysm 120.

Once properly disposed within aneurysm 120, flow diverter 300 may be pushed out of the distal end of microcatheter 200 to allow the frame of wires 420 to self-expand under bias from the wires 420 themselves (see, e.g., FIGS. 4A-5C), for example as illustrated in FIG. 3.

As illustrated in FIG. 3, intrasacular flow diverter 300 may comprise a collapsed or collapsible, substantially spherical frame, formed by wires 420 (see, e.g., FIGS. 4A-4D), that is configured to expand and substantially conform to a shape of an inside surface of aneurysm 120. Diverter 300 comprises a first portion 310, having a first desired porosity, configured to be disposed within and/or directly against a neck of aneurysm 120 and, thereby, protect the neck of aneurysm 120. Diverter 300 may further comprise a second portion 320, adjacent to first portion 310, having a second desired porosity that is greater than the first porosity of first portion 310, and configured to be disposed within aneurysm 120 and distal to the neck of aneurysm 120. Second portion 320 is configured for increased perfusion of blood flow therethrough as compared to the reduced or substantially eliminated perfusion of blood flow through first portion 310. As will be described in more detail in connection with at least FIGS. 4A-4D, these aggregate first and second porosities of first and second portions 310, 320 of diverter 300 may be largely and/or substantially entirely achieved through manipulation of the porosities of corresponding portions of membrane 410 disposed on an entire outer surface of the frame formed by wires 420, rather than through utilization of an ultra-dense collection of a much larger number of wires, as in conventional intrasaccular occluders and/or stents. Accordingly, flow diverter 300 may be configured to address, in some cases, side-wall and/or bifurcation aneurysms having sufficiently wide neck anatomies to contraindicate standard aneurysm treatments.

The cover membrane 310 has porosity, porosity distribution, and permeability characteristics that substantially block the circulating aneurysm inflow 155 and outflow 165 that is illustrated in FIG. 1A. It has been found that this inflow 155 and outflow blocking function can be provided with membranes of surprisingly high porosities and large pore sizes. Generally, to generate a membrane that has low permeability to inflow 155 and outflow 165 in the presence of vessel flow 150, porosity and pore size should be appropriately balanced. Higher total porosities require smaller pore sizes, while lower porosities can have large pore sizes while maintaining the desired inflow 155 and outflow 165 suppression. This may be evaluated in a more quantitative manner by considering the product of median pore size times total fractional porosity as a characterization of cover porosity distribution. For advantageous coiling support cover membranes, this product may be in the range of 0.5 to 50, with 5 to 20 having been found particularly suitable. For example, a suitable membrane may have a total porosity of 0.05 to 0.5 and a median pore size between 10 and 100 microns. In one implementation, a membrane with a porosity of 0.3 to 0.6 and a median pore size between 20 and 30 is utilized. In another implementation, a membrane with a total porosity between 0.05 and 0.15 and a median pore size between 30 and 100 microns is utilized. In another implementation, a membrane with a total porosity between 0.15 and 0.25 and a median pore size between 20 and 80 microns is utilized. In any of the implementations described herein, the pore size range may be from 5 to 400 microns, 5 to 200 microns, or 5 to 100 microns, wherein outliers outside of these ranges that do not significantly contribute the total porosity of the cover (e.g. less than 5% of the total porosity) are ignored. Endothelial ingrowth is enhanced by having relatively small median pore sizes such as 10 to 50 microns and high porosity greater than 0.3, more preferably greater than 0.4.

Relatively high porosity membranes that retain the ability to block significant aneurism inflow 155 and outflow 165 have many advantages. For example, membranes with higher porosities allow the use of wire frames of very high porosity by providing structural support to the device, which allows the production of highly flexible devices that are more easily navigated through complex vasculature to an aneurysm. An electrospun stent cover membrane having porosity and permeability suitable for accomplishing one or more of these objectives is described in paragraphs [0092] through [0103] of US Published Patent Application 2021/0052360, which paragraphs are incorporated by reference herein.

As illustrated in FIG. 4, flow diverter 300 comprises a plurality of wires 420 braided around one another to form a collapsed and/or collapsible (and so also expanded and/or expandible), frame. For example, in some embodiments, each of wires 420 may be coiled into a plurality of collapsible and expandable, substantially helical shape or structure comprising a plurality of loops having successively increasing and then decreasing diameters such that wires 420 form a frame having a predetermined maximum spherical diameter D₁ (e.g., approximately 0.18 inches or 4.5 mm) when fully expanded. In some such embodiments, each wire 420 is offset from adjacent wires 420 by a predetermined spacing L₃. In some embodiments, each of wires 420 may have a predetermined pitch (i.e., each loop or winding of a particular one of wires 420 extends predetermined length). In embodiments where the frame has a substantially spherical maximally expanded shape, this predetermined pitch may be the same distance as the diameter D₁ of the frame, e.g., 0.18 inches or 4.5 mm. In some such embodiments, the spacing L₃ may be determined as the result of dividing the pitch of wires 420, or in appropriate above-mentioned embodiments, diameter D₁, by a number of those wires that are wound in a same direction. For example, in some embodiments according to FIGS. 4A-4D, a subset (e.g., 8, 12 or 16 of the plurality of wires 420 in FIGS. 4A-B, 4C and 4D, respectively) of the plurality of wires 420 are wound in a clockwise direction, while another subset of the plurality of wires 420 (e.g., another 8, 12 or 16 of the plurality of wires 420 in FIGS. 4A-B, 4C and 4D, respectively) are wound in a counter-clockwise direction. Accordingly, where subsets of wires 420 are wound in opposite directions from one another, one subset of wires 420 will overlap the other subset of wires 420 at multiple points along the predetermined maximum-expanded diameter D₁. The above-described geometries of wires 420 advantageously provide for easy, unobstructed expansion of flow diverter 300 within aneurysm 120. In some embodiments, wires 420 comprise super-elastic nitinol. In some other embodiments, a cobalt chromium may be used. In yet other embodiments, a nitinol shape memory alloy may be used. However, the present disclosure is not so limited and wires 420 may comprise any suitably flexible, expandable and compressible material. As most easily seen in any of FIGS. 5A-5D, each of wires 420 may be further coated with a polymer 510 configured to substantially reduce or minimize metal exposure to the blood.

As further illustrated in FIGS. 4A-4D, flow diverter 300 comprises a membrane 410 disposed on an entire outer surface of flow diverter 300. For example, in some embodiments, membrane 410 has a substantially spherical form, as shown in any of FIGS. 4A-4D. At least some portions of membrane 410 may provide a near or substantially impermeable layer that, advantageously, has a very thin construction, as a result of an electrospinning process utilized to form membrane 410 from, for example a polymer. In some embodiments relying on stasis and/or turbulence to clot aneurysm 120, rather than a highly thrombogenic material to produce the clot, membrane 410 may comprise a high-elongation, solvent-dispersible material having good antithrombogenicity properties.

Membrane 410 may also provide support for the underlying expanded frame of wires 420. This support function, along with that of providing the near impermeable layer in at least some portions of membrane 410, work together to allow a reduction in a number of wires 420 (e.g., 8, 12 or 16 wires 420) needed for construction and effective operation, for example compared to the Microvention Web. For example, whereas the Microvention Web relies on the density and close proximity of the many (e.g., 144) individual wires to provide sufficient support to the wall of aneurysm 120 and to also provide sufficiently low porosity to prevent blood flow to aneurysm 120, at least some of the requisite support to the inside surface of aneurysm 120 is provided by membrane 410 in embodiments described anywhere herein. Membrane 410 additionally provides a force that helps expand diverter 300. And a majority of the requisite low permeability of flow diverter 300 is provided by membrane 410.

This reduction in wire number also advantageously improves the navigability of diverter 300 in viva. For example, when comparing same diameters of wire, the smaller number of wires 420 makes diverter 300 easier to navigate into aneurysm 120 than other conventional designs due to the reduced moment of inertia possessed by the smaller number and mass of wires. This is true when diverter 300 is utilized in isolation as well as when diverter 300 is utilized in conjunction with one or more of a stent-based diverters within blood vessel 100 and/or coils disposed within diverter 300 for additional wall contact, radial force, and/or filling of aneurysm 120.

For example, in some embodiments, in order to fill the internal volume of aneurysm 120 additional radial force may be delivered to aneurysm 120 by disposing coils within diverter 300 after diverter 300 is deployed within aneurysm 120. To accomplish this, microcatheter 200 (see FIG. 2) may be repositioned adjacent to diverter 300 after diverter 300 has been deployed within aneurysm 120. A guidewire may be passed through microcatheter 200 and extended sufficiently beyond a distal tip of microcatheter 200 to puncture membrane 410 of diverter 300 and extend within aneurysm 120. Then, microcatheter 200 may be advanced over the guidewire into an interior of diverter 300. Then, traditional coils may be delivered into diverter 300 as necessary.

Another advantage of the use of fewer wires 420, as compared to other conventional designs, is a reduction in stiffness of diverter 300 during the early portions of deployment. This reduction in stiffness also carries over to advantages for diverter 300 in the deployed, extended state, for example, improved wall apposition in irregularly shaped aneurysms. All of these improvements separately and collectively allow for easier tracking into the vasculature and improved delivery and deployment of diverter 300. This is especially true, and advantageous, for applications to smaller and/or tortious blood vessels, such as those of the brain, where turn radii within blood vessels may be very tight and where a ratio of collapsed-to-appropriately deployed radii of diverter 300 may be much smaller than for applications to larger blood vessels, such as the aorta.

Because porosity at any given point on diverter 300 is largely controlled by the porosity and/or permeability of membrane 410, the porosity and/or permeability of membrane 410 can be tailored independently of a number or density of wires 420 to allow very low or substantially no blood flow through areas of membrane 410 configured to be disposed within and/or directly against a neck of aneurysm 120 (see, e.g., the portion of membrane 410 in any of FIGS. 4A-4D that would form or coincide with first portion 310 in FIG. 3) and, thereby, protect the neck of aneurysm 120.

Accordingly, in some embodiments, a density and/or a porosity of membrane 410 may be tuned to have different or variable values at different locations and, thereby, provide for the relatively high porosity of second portion(s) 320 (e.g., a 5-40% porosity) and for the relatively low porosity first portion 310 (e.g., a 0-5% porosity).

Additionally, flow diverter 300 may have an improved applicability for use with coils, or intravascular flow diverters. For example, in the event of inadvertent rupture of an aneurysm while using flow diverter 300, in combination with such coils or intravascular flow diverters, flow diverter 300 provides additional hemostasis via membrane 410 compared to traditional dense wire braids of intrasaccular occluders.

One benefit of traditional coils is that their softness may be tailored or selected to provide minimal disruption or reshaping of irregular shaped aneurysms. Embodiments of diverter 300 described herein comprise far fewer wires 420 than traditional diverters and so are able to provide a similar benefit, at least in that lower force may be applied to the wall of aneurysm 120.

It is also contemplated that wires 420 may have one of a variety of different thicknesses, according to requirements of a desired application. FIGS. 5A-5D illustrates cross-sections of first through fourth example embodiments of intrasacular flow diverter 300 in a collapsed state. Each of the embodiments of FIGS. 5A-5D may be substantially similar to one another, except each utilizes wire 420 having a different diameter and, so, providing a different minimal diameter of flow diverter 300 when in a fully collapsed state.

As illustrated in FIG. 5A, wires 420 may have an outside diameter of approximately 0.0003 inches, and diverter 300 may, accordingly, be configured to have a minimum contracted diameter D₂ (e.g., 0.002 inches) and the maximum expanded diameter D₁.

As illustrated in FIG. 5B, wires 420 may have an outside diameter of approximately 0.0005 inches and diverter 300 may, accordingly, be configured to have a minimum contracted diameter D₃ (e.g., 0.003 inches) and the maximum expanded diameter D₁.

As illustrated in FIG. 5C, wires 420 may have an outside diameter of approximately 0.0007 inches, and diverter 300 may, accordingly, be configured to have a minimum contracted diameter D₄ (e.g., 0.004 inches) and the maximum expanded diameter D₁.

As yet another example, illustrated in FIG. 5D, wires 420 may have an outside diameter of approximately 0.001 inches, and diverter 300 may, accordingly, be configured to have a minimum contracted diameter D₅ (e.g., 0.006 inches) and the maximum expanded diameter D₁.

The example orientation of wires 420 in the collapsed state illustrated in each of FIGS. 5A-5D comprises an innermost subset of 8 threads and an outermost subset of 8 threads. Each of the innermost threads is disposed in direct contact with each of two adjacent threads of the innermost subset and one thread of the outermost subset. A center of each of the outermost threads is radially in-line with both a center of the frame's cross section and a center of the corresponding inner thread with which the outermost thread is in direct contact.

### Example Method(s) of Use

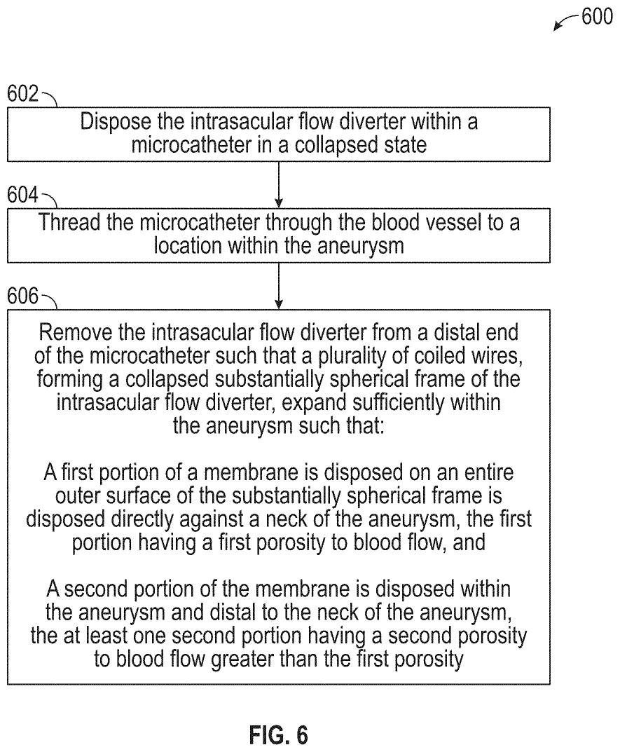

The disclosure now turns to FIG. 6, which illustrates a flowchart 600 related to an example method for utilizing an intrasacular flow diverter, as described anywhere in this disclosure.

Although the method(s) disclosed herein comprise(s) one or more steps or actions for achieving the described method(s), such steps and/or actions may be interchanged with one another, and/or a subset of these steps and/or actions may be used, without departing from the scope of the claims. In other words, unless a specific order of steps or actions is specified, the order and/or use of specific steps and/or actions may be modified without departing from the scope of the claims. One or more additional steps not specifically described herein may also be included.

Step 602 includes disposing the intrasacular flow diverter within a microcatheter in a collapsed state. For example, as previously described in connection with at least FIG. 2, intrasacular flow diverter 300 may be disposed within microcatheter 200 in a collapsed state.

Step 604 includes threading the microcatheter through the blood vessel to a location within the aneurysm. For example, as previously described in connection with at least FIG. 2, microcatheter 200 may be threaded through blood vessel 100 to a location within aneurysm 120.

Step 606 includes removing the intrasacular flow diverter from a distal end of the microcatheter such that a plurality of coiled wires forming a collapsed substantially spherical frame of the intrasacular flow diverter expand sufficiently within the aneurysm that a first portion of a membrane disposed on an entire outer surface of the substantially spherical frame is disposed directly against a neck of the aneurysm, the first portion having a first porosity to blood flow, and a second portion of the membrane is disposed within the aneurysm and distal to the neck of the aneurysm, the at least one second portion having a second porosity to blood flow greater than the first porosity.

For example, as previously described in connection with at least one of FIGS. 2-5C, intrasacular flow diverter 300 may be removed from a distal end of microcatheter 200 such that wires 420, forming a collapsed substantially spherical frame of intrasacular flow diverter 300, expand sufficiently within aneurysm 120 that first portion 310 of membrane 410 disposed on an entire outer surface of the substantially spherical frame is disposed directly against a neck of aneurysm 120, and second portion 320 of membrane 410 is disposed within aneurysm 120 and distal to the neck of aneurysm 120. First portion 310 has a first porosity to blood flow and second portion 320 has a second porosity to blood flow greater than the first porosity.

In some embodiments, each of wires 420 are wound in a substantially helical shape. In some embodiments, each of wires 420 includes a plurality of helical loops, each having predetermined pitch that is substantially equal to maximum expanded diameter D₁ of the substantially spherical frame. In some embodiments, the plurality of helical loops have successively increasing and then decreasing diameters to, thereby, form the substantially spherical structure. In some embodiments, a first subset of wires 420 are wound in a clockwise direction and a second subset of wires 420 are wound in a counterclockwise direction. In some embodiments, each wire 420 is offset from at least one adjacent wire 420 by a predetermined spacing L₁. In some embodiments, wires 420 are one of 8 wires, 12 wires and 16 wires. In some embodiments, wires 420 are configured to self-expand under a self-bias.

In some embodiments, the first porosity of first portion 310 of intrasacular flow diverter 300 is within the range of approximately 0-5%. In some embodiments, the second porosity of second portion 320 of intrasacular flow diverter 300 is within the range of approximately 5-40%.

In some embodiments, each of wires 420 has a diameter of one of approximately 0.0003 inches, approximately 0.0005 inches, 0.0007 inches and 0.001 inches. In some embodiments, intrasacular flow diverter 300 is configured to have a minimum outside diameter D₂, D₃, D₄, D₅ of one of approximately 0.002 inches, approximately 0.003 inches, approximately 0.004 inches, and approximately 0.006 inches when the substantially spherical structure is fully collapsed. In some embodiments, intrasacular flow diverter 300 is configured to have a maximum outside diameter D₁ of approximately 0.18 inches when the substantially spherical structure is fully expanded.

In some embodiments, a substantial majority of an aggregate porosity of intrasacular flow diverter 300, at first and second portions 310, 320 of membrane 410, is derived from the first and second porosities of membrane 410 at respective first and second portions 310, 320.

In some embodiments, each of wires 420 comprises a super-elastic nitinol. In some embodiments, membrane 410 comprises a polymer. In some embodiments, membrane 410 is electrospun from a polymeric material. In some embodiments, each of wires 420 is coated with polymer 510 configured to substantially reduce exposure of wires 420 with the blood.

In some embodiments, wires 420 include a number of wires sufficiently low such that wires 420 expanding sufficiently within aneurysm 120 cause apposition of membrane 410 to an irregular shape of an inside surface of aneurysm 120.

### Example Methods of Manufacture

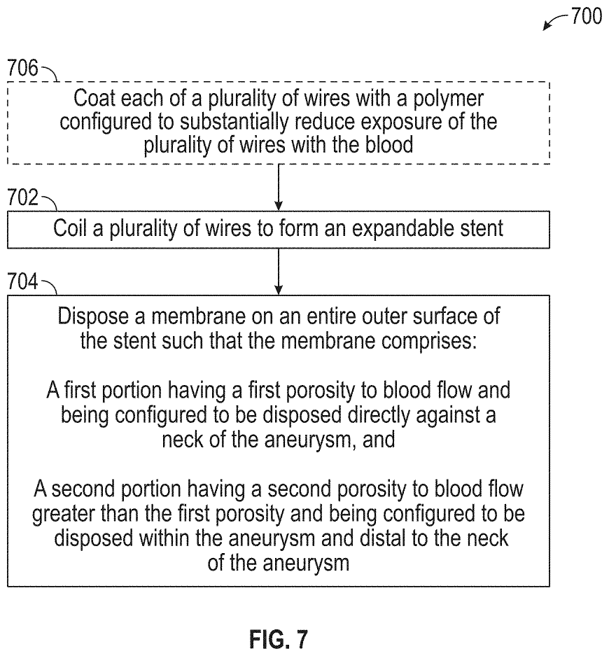

The disclosure now turns to FIG. 7, which illustrates a flowchart 1000 related to an example method of manufacturing an intrasacular flow diverter, as described anywhere in this disclosure.

Although the method(s) disclosed herein comprise(s) one or more steps or actions for achieving the described method(s), such steps and/or actions may be interchanged with one another, and/or a subset of these steps and/or actions may be used, without departing from the scope of the claims. In other words, unless a specific order of steps or actions is specified, the order and/or use of specific steps and/or actions may be modified without departing from the scope of the claims. One or more additional steps not specifically described herein may also be included.

Step 702 includes coiling a plurality of wires to form a collapsible, substantially spherical frame configured to expand within, and substantially conform to a shape of an inside surface of, the aneurysm.

For example, as previously described in connection with at least one of FIGS. 2-5C, wires 420 may be coiled to form a collapsible, substantially spherical frame configured to expand within, and substantially conform to a shape of an inside surface of, aneurysm 120.

In some embodiments, coiling the plurality of wires to form the collapsible, substantially spherical frame comprises winding each of wires 420 in a substantially helical shape. In some embodiments, winding each of the plurality of wires wound in a substantially helical shape comprises winding each of wires 420 to form a plurality of helical loops, each having a predetermined pitch that is substantially equal to a maximum expanded diameter D₁ of the substantially spherical frame. In some embodiments, the plurality of helical loops are wound to have successively increasing and then decreasing diameters to, thereby, form the substantially spherical structure. In some embodiments, a first subset of wires 420 are wound in a clockwise direction and a second subset of wires 420 are wound in a counterclockwise direction. In some embodiments, each wire 420 is offset from at least one adjacent wire 420 by a predetermined spacing L₁. In some embodiments, wires 420 include one of 8 wires, 12 wires and 16 wires. In some embodiments, wires 420 are configured to self-expand under self-bias. In some embodiments, each of the plurality of wires has a diameter of one of approximately 0.0003 inches, approximately 0.0005 inches, 0.0007 inches and 0.001 inches. In some embodiments, each of the plurality of wires comprises a super-elastic nitinol. In some embodiments, wires 420 include a number of wires sufficiently low to allow for apposition of membrane 410 to an irregular shape of an inside surface of aneurysm 120 when intrasacular flow diverter 300 is properly disposed within aneurysm 120.

In some embodiments, intrasacular flow diverter 300 is configured to have a minimum outside diameter D₂, D₃, D₄, D₅ of one of approximately 0.002 inches, approximately 0.003 inches, approximately 0.004 inches, and approximately 0.006 inches when the substantially spherical structure is fully collapsed. In some embodiments, intrasacular flow diverter 300 is configured to have a maximum outside diameter D₁ of approximately 0.18 inches when the substantially spherical structure is fully expanded.

Step 704 includes disposing a membrane on an entire outer surface of the frame such that the membrane comprises a first portion having a first porosity to blood flow, the first portion configured to be disposed directly against a neck of the aneurysm, and a second portion having a second porosity to blood flow greater than the first porosity, the second portions configured to be disposed within the aneurysm and distal to the neck of the aneurysm.

For example, as previously described in connection with at least one of FIGS. 2-5C, membrane 410 may be disposed on an entire outer surface of the frame such that membrane 410 comprises first portion 310 having a first porosity to blood flow and configured to be disposed directly against a neck of aneurysm 120, and second portion 320 having a second porosity to blood flow greater than the first porosity and configured to be disposed within aneurysm 120 and distal to the neck of aneurysm 120.

In some embodiments, the first porosity is within the range of approximately 0-5%. In some embodiments, the second porosity is within the range of approximately 5-40%. In some embodiments, a substantial majority of an aggregate porosity of intrasacular flow diverter 300, at first and second portions 310, 320 of membrane 410, is derived from the first and second porosities of membrane 410 at respective first and second portions 310, 320. In some embodiments, membrane 410 is electrospun from a polymeric material.

In some embodiments, a method related to flowchart 700 may include an optional step 706, including coating each of the plurality of wires with a polymer configured to substantially reduce exposure of the plurality of wires with the blood.

For example, as previously described in connection with at least one of FIGS. 2-5C, each of wires 420 may be coated with a polymer configured to substantially reduce exposure of wires 420 with the blood.

General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. An intrasacular flow diverter for treating an aneurysm of an intracranial blood vessel, the intrasacular flow diverter comprising:
one or more wires forming a frame, wherein the frame has a collapsed configuration and an expanded configuration, wherein the frame is configured to transition in use from the collapsed configuration to a deployed configuration that substantially conforms to a shape of an inside surface of a sac of the aneurysm;
an electrospun cover disposed on at least a portion of the frame, the cover comprising:
a first portion configured to be disposed against a neck of the aneurysm, the first portion of the cover having pores formed therein defining a first porosity thereof; and
a second portion having a second porosity to blood flow greater than the first porosity, the second portion configured to be disposed within the sac of aneurysm distal to the neck of the aneurysm.

2. The intrasacular flow diverter of claim 1, wherein the frame comprises each of the one or more wires wound in a substantially helical shape.

3. The intrasacular flow diverter of claim 2, wherein a plurality of helical loops have successively increasing and then decreasing diameters to, thereby, form a substantially spherical structure.

4. The intrasacular flow diverter of claim 1, wherein the plurality of wires is one of 8 wires, 12 wires and 16 wires.

5. The intrasacular flow diverter of claim 1, wherein the one or more wires are configured to self-expand under a bias from the one or more wires.

6. The intrasacular flow diverter of claim 1, wherein the intrasacular flow diverter is configured to be disposed, in a collapsed form, within a microcatheter that is configured to be threaded through the blood vessel to a location within the aneurysm.

7. The intrasacular flow diverter of claim 1, wherein the first porosity is less than 0.05.

8. The intrasacular flow diverter of claim 1, wherein the second porosity is greater than 0.05.

9. The intrasacular flow diverter of claim 1, wherein each of the plurality of wires has a diameter of one of approximately 0.0003 inches, approximately 0.0005 inches, 0.0007 inches and 0.001 inches.

10. The intrasacular flow diverter of claim 1, configured to have a minimum outside diameter of one of approximately 0.002 inches, approximately 0.003 inches, approximately 0.004 inches, and approximately 0.006 inches when the substantially spherical structure is fully collapsed.

11. The intrasacular flow diverter of claim 1, wherein the intrasacular flow diverter is configured to have a maximum outside diameter of approximately 0.18 inches when the substantially spherical structure is fully expanded.

12. The intrasacular flow diverter of claim 1, wherein a substantial majority of an aggregate porosity of the intrasacular flow diverter, at the first and second portions of the membrane, is derived from the first and second porosities of the membrane at the respective first and second portions.

13. The intrasacular flow diverter of claim 1, wherein each of the one or more wires comprises a super-elastic nitinol.

14. The intrasacular flow diverter of claim 1, wherein the membrane comprises a polymer.

15. A method of utilizing an intrasacular flow diverter to treat an aneurysm of a blood vessel, the method including:
disposing the intrasacular flow diverter within a microcatheter in a collapsed state;
threading the microcatheter through the blood vessel to a location within the aneurysm; and
removing the intrasacular flow diverter from a distal end of the microcatheter such that a plurality of coiled wires forming a collapsed substantially spherical frame of the intrasacular flow diverter expand sufficiently within the aneurysm that:
a first portion of a membrane disposed on at least a portion of the outer surface of the substantially spherical frame is disposed directly against a neck of the aneurysm, the first portion having a first porosity to blood flow, and
a second portion of the membrane is disposed within the aneurysm and distal to the neck of the aneurysm, the second portion having a second porosity to blood flow greater than the first porosity.

16. The method of claim 15, wherein the plurality of wires are configured to self-expand under a bias from the plurality of wires.

17. The method of claim 15, wherein the first porosity is less than 0.05.

18. The method of claim 15, wherein the second porosity is between 0.05 and 0.4.
